(19) United States
(12) Patent Application Publication
(10) Pub. No.: US 2024/0044912 A1
(43) Pub. Date: Feb. 8, 2024

(54) MEANS FOR ANTIBODY CHARACTERIZATION

(71) Applicants: THE REGENTS OF THE UNIVERSITY OF MICHIGAN, Ann Arbor, MI (US); SANOFI, Paris (FR)

(72) Inventors: Yatin GOKARN, Lexington, MA (US); Jonathan KINGSBURY, Bridgewater, NJ (US); Charles G. STARR, Ann Arbor, MI (US); Peter M. TESSIER, Ann Arbor, MI (US)

(21) Appl. No.: 18/269,118

(22) PCT Filed: Jan. 5, 2022
(86) PCT No.: PCT/EP2022/050148

§ 371 Date: Jun. 22, 2023

Related U.S. Application Data
(60) Provisional application No. 63/134,050, filed on Jan. 5, 2021.

(30) Foreign Application Priority Data
Mar. 30, 2021 (EP) 21315056.8

(51) Int. Cl. G01N 33/68; B82Y 15/00; G01N 33/543

(52) U.S. Cl. CPC G01N 33/6854; B82Y 15/00; G01N 33/54346

(57) ABSTRACT

The present disclosure provides means such as uses, nanoparticles, solutions, methods, kits and systems for screening target proteins for self-association properties (viscosity and opalescence) in ultra-dilute solutions. They provide means for screening a large number of target proteins at orders of magnitude lower concentrations than end-use formulations.

### FIELD

The present disclosure provides means such as uses, nanoparticles, solutions, methods, kits and systems useful for detecting self-association characteristics of proteins (e.g., therapeutic antibodies) using ultra-dilute solution measurements.

### BACKGROUND

Monoclonal antibodies (mAbs) are among the most successful pharmaceutical modalities, used to treat a wide array of diseases. Despite their success, mAbs are prone to developability issues that pose major manufacturing, stability, and delivery challenges. In particular, the development of mAbs intended for subcutaneous administration can be hindered by the need for high concentration formulations, in which poor solution behavior in the form of high viscosity, opalescence, phase separation, and aggregation are often limiting. While intravenous administration of mAbs in hospital settings is routine, subcutaneous administration, being minimally invasive, ensures greater patient compliance and is readily adaptable in settings with inadequate medical infrastructures. Rapid selection of developable therapeutic mAb candidates is always desired, but this urgency becomes even more acute when combating infectious disease pandemics. When developability issues do arise, resource intensive mitigation strategies must be employed which invariably cause delays and do not guarantee success. As such, it is far more efficient to select mAbs with favorable solution properties early with significant emphasis being placed on identifying variants with drug-like properties at the earliest stages of discovery.

### SUMMARY

Disclosed herein is, in a first aspect, the use of a positively-charged polymer to stabilize a nanoparticle comprising a capture agent on the surface.

Disclosed herein is, in a second aspect, a nanoparticle comprising on the surface a capture agent and a positively-charged polymer.

Disclosed herein is, in a third aspect, a solution comprising a plurality of the nanoparticle of the second aspect.

Disclosed herein is, in a fourth aspect, a kit comprising a nanoparticle and a positively-charged polymer.

Disclosed herein is, in a fifth aspect, a method for determining the tendency of a target protein to self-associate, comprising the steps of
(i) capturing the target protein in a solution as defined in the third aspect,
(ii) determining the color of the solution,
wherein a change in the color of the solution compared to a control solution as defined in the third aspect without a target protein indicates a tendency of the target protein to self-associate.

Disclosed herein are, in a sixth aspect, methods for screening a target protein in dilute concentrations for self-association properties, comprising: adsorbing a capture agent and a positively-charged polymer onto a surface (e.g., nanoparticle (e.g., a metal nanoparticle)) to form a capture agent conjugate; incubating the capture antibody conjugate with the target protein in a solution (e.g., buffer) to form a target protein conjugate; measuring the absorbance of light of the target protein conjugate at multiple wavelengths ranging from 450 nm to about 750 nm; and identifying a plasmon wavelength as the wavelength at which there is maximal absorbance by the target protein conjugate.

Disclosed herein are, in a seventh aspect, systems or kits comprising one or more of each of a capture agent, a positively-charged polymer, a capture surface (e.g., a metal nanoparticle), a solution (e.g., buffer), a target protein, at least one calibration protein, and a spectrophotometer.

Other aspects and embodiments of the disclosure will be apparent in light of the following detailed description and accompanying figures.

### BRIEF DESCRIPTION OF THE DRAWINGS

FIG. 1 is a schematic of an exemplary research and development process for monoclonal antibodies. Antibody discovery campaigns begin with the identification and verification of thousands of candidate antibodies that recognize the target biomolecule. The large pool is condensed to the order of hundreds during the optimization phase, in which candidates undergo affinity maturation and humanization. During these early stages, minute quantities (ng-μg range) of material are available, rendering the assessment of developability quality attributes such as viscosity, opalescence, and aggregation infeasible. Because of this, it is not uncommon for molecules with poor developability attributes to advance into clinical development, greatly increasing the amount of time and resources spent on generating a viable drug product. The assessment of key developability attributes during lead optimization using ultra low consumption, high throughput assays, such as the CS-SINS approach described herein would facilitate the advancement of antibody candidates with a reduced risk for complications during manufacture and formulation in later stages of development, accelerating timelines and reducing associated resource expenditures.

FIGS. 2A-2D shows the evaluation of the stabilization of antibody-gold conjugates using polylysine. Immunogold conjugates from standard AC-SINS implementations (FIG. 2A, left) were assessed for stability under common formulation and process conditions (10 mM acetate and histidine buffers, pH 4.0-6.5). Through dynamic light scattering measurements, rapid aggregation of the conjugates was observed beginning at pH 5.0 and continuing through the relevant conditions up to pH 6.5 (FIG. 2B, left). A concomitant shift in the plasmon wavelength of the aggregated conjugates was observed, measured by absorbance spectroscopy (FIG. 2C, left). The zeta potential was measured across the pH range and a consistent decline in potential was observed as pH increased from 4.0, crossing zero near pH 5.75, and turning negative thereafter (FIG. 2D, left). To achieve immunogold conjugate stability in the formulation space, a small amount (3% by mass) of polylysine, a highly charged biopolymer, was incorporated (FIG. 2A, center). Particle stabilization was observed up to pH 6.0 using both DLS measurements of particle size (FIG. 2B, middle) and optical measurements of plasmon wavelength (FIG. 2C, middle). The zeta potential of the conjugates remained positive across the assayed pH range (FIG. 2D, middle). However, the absolute value of the potential near pH 6.0 was near zero, which is associated with aggregation in analogous experiments with non-stabilized conjugates. Stabilization increased as polylysine was increased throughout the range of 3%-15%, as demonstrated by a highly stabilized 15% polylysine condition (FIG. 2A, right). These conjugates were stable across the relevant pH range, as indicated by DLS size measurements (FIG. 2B, right) and plasmon wavelength (FIG. 2C, right). The zeta potential of the highly stabilized particles was positive across the measured range (FIG. 2D, right). FIG. 2E is an exemplary graph of the absorbance as a function of wavelength used to determine the plasmon wavelength of the nanoparticles.

FIGS. 3A-3H show the application of charge-stabilized immunogold conjugates for the assessment of weak protein-protein interactions. A diverse panel of 56 monoclonal antibodies was employed to assess the utility of charge-stabilized immunogold conjugates in identifying antibody self-interactions. The panel represented a faithful recapitulation of the clinical antibody landscape, as evidenced by similarities in the biophysical properties to a dataset of 500 molecules extracted from the Therapeutic Antibody Database (TABS) (FIGS. 3A-3D). The robustness of the calibration approach (described in methods) was demonstrated by comparing the results of experiments performed with varying concentrations of polylysine (3% vs. 10%). An excellent correlation was observed between the two conditions, demonstrating that the assay is resistant to influence from small changes in polylysine concentration that may occur due to variability in reagent preparation (FIG. 3E). CS-SINS scores were compared to direct measurements of critical developability properties (See Kingsbury, J. S. et al. Sci Adv 6, eabb0372, doi:10.1126/sciadv.abb0372 (2020), incorporated herein by reference in its entirety), which empirically established that mAbs with viscosity >30 cP or opalescence >12 NTU portend issues in manufacture and formulation during clinical development. Applying these benchmarks, it was shown that using a CS-SINS threshold of 0.35 led to the identification of well behaved (viscosity <30 cP and opalescence <12 NTU), viscous (>30 cP) or opalescent (>12 NTU), or problematic solution behavior in >85% of the mAb panel (FIGS. 3F-3H).

FIG. 4 shows the methodology of the CS-SINS calibration process. CS-SINS measurements are calibrated and evaluated using two tests. Only CS-SINS measurements that pass both tests should be accepted.

FIGS. 5A-5C show an exemplary calibration of CS-SINS measurements. Examples of experiments that passed (FIG. 5A) and failed (FIGS. 5B-5C) the calibration process due to slow mixing of the goat anti-human Fc antibody with the gold particles (FIG. 5B) or the addition of 50% of the target mAb concentration (FIG. 5C). To pass Test #1, the antibody-gold conjugates display plasmon wavelengths less than 534 nm (human polyclonal antibody) and 533 nm (NIST mAb). To past Test #2, the conjugates display linear fit parameters for the calibration panel versus the reference panel of mAbs that are within 10% of the ideal values (1 for slope, 0 for intercept and 1 for R²). The CS-SINS scores are calculated as described herein.

FIGS. 6A and 6B demonstrate that calibrated CS-SINS results are strongly correlated for conjugates prepared using different polylysine concentrations. A robust calibration protocol was developed to normalize results from experiments with varying amounts of polylysine. Plasmon shift measurements for a panel of mAbs using goat anti-human Fc antibody conjugates stabilized with polylysine at polylysine/IgG mass fractions of 0.03 (97% IgG) and (90% IgG) (FIG. 6A). FIG. 6B is a graph of the correlation between the CS-SINS scores for the panel of mAbs measured at the two polylysine/IgG mass fractions for the plasmon measurements reported in FIG. 6A using the calibration detailed in FIGS. 4 and 5.

FIGS. 7A-7C show that CS-SINS measurements are strongly correlated with diffusion interaction parameters in an IgG subclass-specific behavior. CS-SINS scores (0.01 mg/mL) are well correlated with kD measurements (1-10 mg/mL) (FIG. 7A, top). CS-SINS scores are even better correlated kD measurements for the subset of IgG1/IgG2 anti-bodies (FIG. 7A, middle) and less correlated for the subset of IgG4 antibodies (FIG. 7A, bottom). The solution behavior in the context of subclass reveals that the proposed 0.35 threshold value for CS-SINS measurements effectively identifies all of the poorly behaved molecules in the IgG1/IgG2 subclass grouping (FIG. 7B). The solution behavior for IgG4s is more difficult to predict as the majority are prone to high opalescence but do not always respond in the CS-SINS assay (FIG. 7C).

FIGS. 8A-8C are graphs of the evaluation of the use of polylysine-stabilized gold conjugates for measuring mAb self-association in histidine formulations (pH 6). Gold particles were first coated with goat anti-human Fc IgG and polylysine (≥70 kDa) (FIG. 8A), goat non-specific IgG and polylysine (FIG. 8B), or only polylysine (FIG. 8C), and then the conjugates were incubated with a panel of mAbs with different levels of self-association. To evaluate the dependence of the plasmon shifts on the amount of mAb adsorbed, the conjugates (after the first step conjugation step) were incubated with a constant concentration of human antibody (0.01 mg/mL) that was composed of only human polyclonal antibody (0% mAb), only human mAb (100% mAb) or combinations thereof. The reported plasmon shifts are relative to human polyclonal antibody. In FIGS. 8A and 8B, the mass ratio of polylysine to IgG was 0.03, and the concentrations of polylysine and IgG used during conjugation were 0.012 and 0.388 mg/mL, respectively. In FIG. 8C, the concentration of polylysine used during conjugation was 0.4 mg/mL.

FIGS. 9A and 9B are graphs showing the effect of polylysine size on CS-SINS measurements of plasmon shifts in histidine formulations (pH 6). Conjugates were prepared by co-adsorbing goat anti-human Fc antibody with different size polylysine polymers, and then the conjugates were used to measure plasmon shifts for a panel of human mAbs. FIG. 7A shows the correlation between plasmon shifts measured using 30-70 kDa polylysine (0.03 polylysine/IgG mass ratio) and 70 kDa poly-lysine (0.03 polylysine/IgG mass ratio). FIG. 7B shows the correlation between plasmon shifts measured using 15-30 kDa poly-lysine polymers (0.10 polylysine/IgG mass ratio) and #70 kDa polylysine polymers (0.03 polylysine/IgG mass ratio).

### DETAILED DESCRIPTION

The present disclosure provides methods for predicting problematic characteristics (e.g., self-association) of protein formulations using small protein amounts in ultra-dilute solutions.

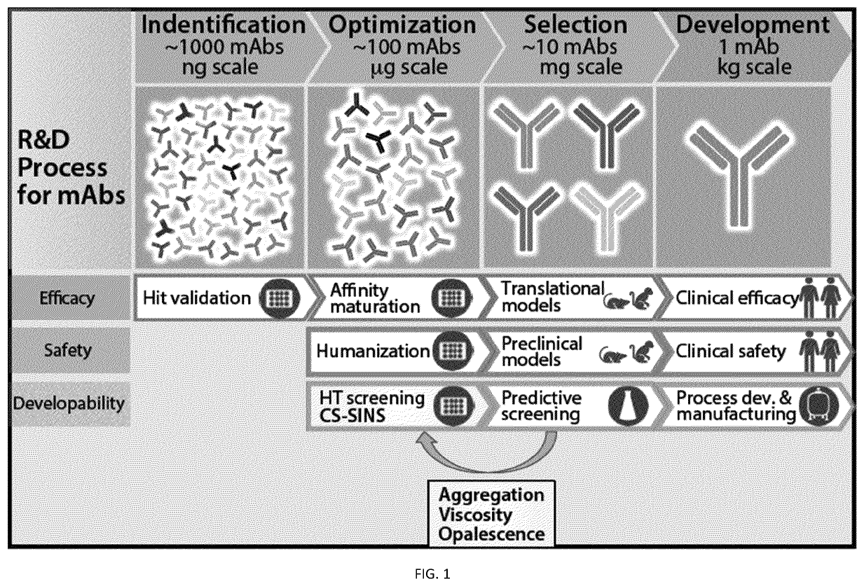

Antibodies with low levels of self-association present a reduced risk of unfavorable solution behavior during manufacturing, formulation, and delivery at high concentrations. While dilute-solution interactions present an effective approach for predicting antibody solution behavior, it is challenging to employ relevant screening methods for large numbers of antibodies during early discovery (FIG. 1). Traditional techniques require relatively concentrated protein solutions (>1 mg/mL) for making such measurements. This single challenge related to antibody concentration has prevented large and systematic analyses of antibody self-association during antibody lead optimization (FIG. 1). Assaying antibody interactions at much lower concentrations (1-10 μg/mL) in a manner predictive of high concentration solution behavior (e.g., viscosity at 150 mg/mL) would enable the selection of well-behaved mAbs from a much larger pool of candidates in early discovery.

A previously used approach (affinity-capture nanoparticle spectroscopy, AC-SINS) involved adsorbing anti-human capture antibodies on gold nanoparticles (20 nm) and using the conjugates to capture human mAbs of interest. The capture of multiple mAbs in proximity amplified colloidal interactions and led to sensitive detection of antibody self-interactions via measurable changes in optical properties inherent to gold nanoparticles. However, a key limitation of AC-SINS was that it was not compatible with process streams and formulation conditions in the pH 4.5-7 range and low ionic strength, commonly employed during antibody development. Gold nanoparticles coated with capture (anti-human) antibodies were unstable under such conditions and readily aggregate.

Described herein is development of systems and methods referred to as charge-stabilized self-interaction nanoparticle spectroscopy (CS-SINS) that overcame the limitations of AC-SINS by stabilizing the antibody-gold conjugates using positively-charged polymers. Moreover, CS-SINS was capable of robustly evaluating mAb weak, self-interactions at ultra-dilute concentrations (10 μg/mL) that predict problematic high viscosity and opalescence of antibodies at four orders of magnitude higher concentrations (150 mg/ml).

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “antibody,” as used herein, refers to a protein that is endogenously used by the immune system to identify and neutralize foreign objects, such as bacteria and viruses. Typically, an antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other. The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)). The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H}, domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

A “spectrophotometer” is any variety of instrument that measure light absorption and/or transmission at a variety of light wavelengths. A spectrophotometer is most commonly applied to ultraviolet (185-400 nm), visible (400-700 nm), and infrared (700-15000 nm) radiation, but some spectrophotometers can interrogate wide swaths of the electromagnetic spectrum, including x-ray, ultraviolet, visible, infrared, and/or microwave wavelengths. Spectrophotometers have a light source, a monochromator, a wavelength selector, a sample holder, and a detector. Any spectrophotometer capable of processing solution-based samples may be used, including those with cuvette sample holders, plater readers, and the like.

A “polypeptide”, “protein,” or “peptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

Positively-charged polymers can be any of a variety of compounds having a net positive charge. Positively-charged polymers useful in the present invention include positively charged peptides and proteins, both naturally occurring and synthetic, as well as polyamines, carbohydrates or synthetic polycationic polymers. The positively-charged polymers may be linear or branched polymers, and may have interconnection between repeating units in addition to the main chain linkages. Positively-charged polymer compositions may have any level of polydispersity from substantially monodisperse to substantially polydisperse. A substantially monodisperse composition comprises polymer molecules, substantially all of which have the same chain length. A substantially polydisperse composition comprises polymer molecules with a variety of chain lengths (and hence molecular weights).

The concentration of the positively-charged polymer may vary but is directed to those concentrations which prevent self-aggregation of the capture agent conjugate in the absence of a target protein. Aggregation of the capture agent conjugate can be measured under a variety of solution conditions (pH, conjugate concentration, temperature, ionic strength) by a number of methods known in the art, including for example, dynamic light scattering as demonstrated herein.

The concentration of the positively-charged polymer may be greater than about 0.1% by mass of positively-charged polymer and capture agent (i.e. 0.1% positively-charged polymer and 99.9% capture agent), greater than about 1% by mass of positively-charged polymer and capture agent, greater than about 3% by mass of positively-charged polymer and capture agent, greater than about 5% by mass of positively-charged polymer and capture agent, greater than about 10% by mass of positively-charged polymer and capture agent, greater than about 15% by mass of positively-charged polymer and capture agent, greater than about 20% by mass of positively-charged polymer and capture agent, greater than about 30% by mass of positively-charged polymer and capture agent, greater than about 40% by mass of positively-charged polymer and capture agent. In some embodiments, the positively-charged polymer is added at a concentration greater than 3% by mass of positively-charged polymer and capture agent.

The concentration of the positively-charged polymer may be between about 0.1% to about 50% by mass of positively-charged polymer and capture agent. The concentration of the positively-charged polymer may be between about 0.1% to about 50%, between about 1% to about 50%, between about 3% to about 50%, between about 5% to about 50%, between about 10% to about 50%, between about 20% to about 50%, between about 30% to about 50%, between about 0.1% to about 40%, between about 1% to about 40%, between about 3% to about 40%, between about 5% to about 50%, between about 10% to about 50%, between about 20% to about 50%, between about 30% to about 50%, between about 0.1% to about 30%, between about 1% to about 30%, between about 3% to about 30%, between about 5% to about 30%, between about 10% to about 30%, between about 20% to about 30%, between about to about 20%, between about 1% to about 20%, between about 3% to about 20%, between about 5% to about 20%, between about 10% to about 20%, between about 0.1% to about 15%, between about 1% to about 15%, between about 3% to about 15%, between about 5% to about 15%, between about 10% to about 15%, between about 0.1% to about 10%, between about 1% to about 10%, between about 3% to about 10%, between about 5% to about 10%, between about 0.1% to about 5%, between about 1% to about 5%, between about 3% to about 5%, between about 0.1% to about 3%, between about 1% to about 3%, or between about to about 1% by mass of positively-charged polymer and capture agent. In some embodiments, the positively-charged polymer is added at a concentration greater than 3% by mass of positively-charged polymer and capture agent. In some embodiments, the positively-charged polymer is added at a concentration between about 3% and about 15% by mass of positively-charged polymer and capture agent.

Positively-charged polymers can have a wide range of molecular weights. In some embodiments, a positively-charged polymers can have a molecular weight greater than about kDa, greater than about 15 kDa, greater than about 20 kDa, greater than about 25 kDa, greater than about 30 kDa, greater than about 40 kDa, greater than about 50 kDa, or greater than about 60 kDa, greater than about 70 kDa, greater than about 80 kDa, greater than about kDa, greater than about 100 kDa, greater than about 150 kDa, greater than about 200 kDa, or greater. In other embodiments, the positively-charged polymers can have a molecular weight between 10-500 kDa, between 10-250 kDa, between 10-200 kDa, between 15 and 70 kDa, between 30 and 70 kDa, or between 15 and 30 kDa. However, other sizes may be used which prevent self-aggregation of the capture agent conjugate. Molecular weights can be determined by those of ordinary skill in the art by methods such as size-exclusion chromatography and/or multi-angle laser light scattering techniques.

In certain embodiments of the invention the positively-charged polymer may fall under the class of synthetic polypeptides, also known as polyamino acids. A synthetic polypeptide may be a homopolymer of one of the positively charged (i.e., basic) amino acids such as lysine, arginine, or histidine, or a heteropolymer of two or more positively charged amino acids. In some embodiments, the polycation may be polylysine (e.g., poly-D-lysine, poly-L-lysine, and poly-DL-lysine), polyarginine, and polyhistidine, in particular polylysine. In addition, the polymer may comprise one or more positively charged non-standard amino acids such as ornithine, 5-hydroxy lysine, and the like. Or, the polypeptide may be functionalized with other groups, such as poly(y-benzyl-L-glutamate). Any of the combination amino acids can be polymerized into linear, branched, or cross-linked chains. Such polycationic polypeptides may contain at least 50 amino acid residues, at least 100 amino acid residues, at least 200 amino acid residues, at least 300 amino acid residues, at least 500 amino acid residues, at least 750 amino acids, at least 1000 amino acids, at least 2000 amino acids, at least 3000 amino acids, at least 4000 amino acids or more (e.g., from about 50 to about 500 amino acid residues, from about 50 to about 1000 amino acid residues, or from about 100 to about 1000 amino acid residues). Synthetic polypeptides can be produced by methods known to those of ordinary skill in the art, for example, by chemical synthetic methods or recombinant methods. In select embodiments, the positively-charged polymer is polylysine of greater than or equal to about 70 kDa. In select embodiments, the positively-charged polymer is polylysine added at a concentration of 3-15% weight to volume.

In some embodiments, the positively-charged polymers comprise synthetic polycationic polymers, including but not limited to polyethylenimine (PEI), polyamidoamine (PAMAM), and the like.

The term “stabilizing” with regard to nanoparticles refers in particular to a prevention or at least reduction of aggregation of nanoparticles. “Aggregation” refers to formation of assemblages in a suspension and represents a mechanism leading to the functional destabilization of colloidal systems. During this process, particles dispersed in the liquid phase stick to each other, and spontaneously form irregular particle assemblates, flocs, or agglomerates. This phenomenon is also referred to as coagulation or flocculation and such a suspension is also called unstable.

As used herein, the term “nanoparticle” refers to small particles having a size (e.g. diameter) on the scale of 0.001 μm to 1 μm. Nanoparticles may be in the form of spheres, rods, chains, stars, flowers, reefs, whiskers, fibers, boxes, and the like. The size refers to the largest distance from one point of the nanoparticle to another, e.g. for spheres it is the diameter.

Nanoparticles may comprise any material, including metals, semiconductor materials, magnetic materials, and combinations of materials. Specifically envisaged with regard to the present disclosure are metal nanoparticles, wherever referred to herein. Metal nanoparticles, e.g., gold or silver nanoparticles, are inherently suitable for surface plasmon resonance-based assays, since metal spheres have free electrons on their surface that can interact with the electric field from incident light, resulting in a strong absorbance spectrum. In some embodiments, the nanoparticle comprises a gold nanoparticle, has a gold surface or consists of gold. The size of the nanoparticle may affect the intensity and wavelength of maximum absorbance. In some embodiments, the nanoparticles have a diameter from 1 nm to 1000 nm, e.g., 1 nm to 200 nm. In some embodiments, the nanoparticle is 5 nm to 50 nm in size (e.g., diameter), such as approximately 20 nm. In exemplary embodiments, the nanoparticle comprises or is a 20 nm gold nanoparticle. The overall quantity of the nanoparticles in the assay may affect the signal to noise measurement. In some embodiments, 20 nm gold nanoparticles are used at a concentration of at least 7.0×10⁹ particles/mL. In some embodiments, 20 nm gold nanoparticles are used at a concentration of 7.0×10⁹-1.5×10⁹ particles/mL (e.g., 1.0×10¹⁰-1.5×10⁹ particles/mL).

A capture agent is any agent that is capable of binding to a target protein (“capturing a target protein”, specifically in a solution as defined in the third aspect). Thus, the nature of the capture agent will depend on the type of target protein. For example, the capture agent may comprise a binding partner of the target protein, either natural or synthetic, including proteins, nucleic acids, carbohydrates, small molecules, or another binding moiety recognized specifically by the target protein. In some embodiments, the capture agent comprises an antibody, or a derivative or fragment thereof, capable of binding the target protein. The capture agent can be directly adsorbed to the nanoparticle, or alternatively, the nanoparticle may comprise a linker that tethers the capture agent to the nanoparticle. In some embodiments, the capture agent is a protein or a protein ligand. In specific embodiments, it is (or comprises) an antibody or a protein comprising an antigen-binding fragment of an antibody. The antibody can in particular be an Fc-specific antibody (i.e. an antibody binding to the Fc portion of another antibody), such as an IgG-Fc-specific antibody, specifically an IgG1-, IgG2- or IgG4-Fc-specific antibody, and more specifically an IgG1- or IgG4-Fc-specific antibody. In some embodiments, the antibody is an anti-human antibody. A target protein may be any protein in which one desires to determine self-association or self-aggregation properties using small quantities of protein in dilute solutions. In some embodiments, the target protein comprises a therapeutic protein. Therapeutic proteins comprise both purified and synthetic proteins useful for the treatment of diseases and disorders in a subject. Therapeutic proteins may include, but are not limited to, antibody-based drugs, Fc fusion proteins, anticoagulants, blood factors, bone morphogenetic proteins, engineered protein scaffolds, enzymes, growth factors, hormones, interferons, interleukins, and thrombolytics. In some embodiments, the target protein comprises an antibody or a fragment or a derivative thereof. In select embodiments, the target protein comprises an antibody, specifically a monoclonal antibody, more specifically a human monoclonal antibody. In one embodiment, the antibody is an IgG antibody, for example an IgG1, IgG2 or IgG4 antibody. In a select embodiment thereof, the antibody is an IgG1 or IgG2 antibody.

The target protein may be at any concentration. In the case of therapeutic proteins, the concentration will likely be considerably more dilute than that of the therapeutic formulation. In some embodiments, the target protein is at a concentration of less than 1 mg/mL (e.g., less than 500 μg/mL, less than 100 μg/mL, less than 50 μg/mL, or less than 10 μg/mL). The target protein may be at a concentration of 1-1000 μg/mL, 1-100 μg/mL, 1-50 μg/mL, 1-20 μg/mL, or 1-10 pg/mL. In some embodiments, the target protein is at a concentration of 1-10 μg/mL. In some embodiments, the target protein is in a solution (e.g., buffer) with a pH value of 3.5-7 (e.g., 4.5-7), specifically 4-6.5 or 4-6. In some embodiments, the solution (e.g. buffer) has low ionic strength.

Ionic strength is a measure of the concentrations of ions in solution. The term “low ionic strength solution” refers to a solution like a buffer with a total buffer agent concentration of less than about 50 mM, whereas high ionic strength buffers are those with a concentration greater than about 100 mM. Buffer agents are well known in the art and can be salts of a weak acid and a weak base. Examples are carbonates, bicarbonates, and hydrogen phosphates. In specific embodiments of the disclosure, the buffer is a histidine and/or acetate buffer or a buffer having substantially the same ionic strength as a histidine and/or acetate buffer. A histidine and/or acetate buffer may have a concentration of less than 50 mM histidine and/or 50 mM acetate, e.g., less than 25 mM histidine and/or 25 mM acetate, specifically about 10 mM histidine and/or 10 mM acetate. “Substantially the same” means ±50%, ±40%, ±30%, ±20%, ±10%, or ±5%.

The term “conjugation” or “conjugating to” refers to the bond of molecules to nanoparticles by chemical, physical or biological means. A conjugation of nanoparticles to a molecule is usually meant when referring to a nanoparticle comprising a molecule on its surface. A specific meaning of the term is “adsorption” or “adsorbing onto”, which includes physisorption (van der Waals forces), chemisorption (covalent bonding) and electrostatic attraction. In a select embodiment, it is physisorption.

The term “self-association” refers to the interaction between the same kind of protein (e.g., antibodies) and a multimer formation. Such multimer formation may return to the monomer by an operation such as dilution. The “tendency” for self-association refers to the ease of self-association, in particular by increasing the concentration of the protein, e.g., above 50 mg/ml, 100 mg/ml or 150 mg/ml (referred to as “high” or “higher” concentration herein). An example of a parameter indicating self-association is the diffusion interaction parameter. The diffusion interaction parameter is an index of self-association calculated using the concentration dependence of the diffusion coefficient obtained by an experimental method. If the value is −12.4 g/mL or more, repulsive force between molecules predominates. If it is less than that, it is reported to be an attractive (self-associative) interaction (Saito et al., Pharm. Res., 2013. Vol. 30 p1263). The diffusion coefficient is an index of the ease of diffusion of molecules in a solution and can be measured by a dynamic light scattering method or the like. More generally, protein self-association may result in poor solution behavior including high viscosity, opalescence, phase separation and/or aggregation.

The term “plasmon wavelength” refers to the wavelength of maximum absorbance.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. USES FOR STABILIZING NANOPARTICLES

In a first aspect, the disclosure relates to the use of a positively-charged polymer to stabilize a nanoparticle comprising a capture agent on the surface.

The capture agent is usually conjugated to the nanoparticle. In specific embodiments, the nanoparticle is comprised in a solution. Accordingly, the use may be for stabilizing nanoparticles, in particular a plurality thereof, in a solution (i.e. for stabilizing a nanoparticle suspension). The solution may be one as defined in the third aspect below.

The use usually comprises conjugating the positively-charged polymer to the nanoparticle before, at the same time, or after conjugating the capture agent to, the nanoparticle, in one specific embodiment at the same time. Concentrations (by mass of positively-charged polymer and capture agent) of the positively-charged polymer can be as described above. In some embodiments, the use comprises determining the stabilization of the nanoparticle by determining a plasmon wavelength of the nanoparticle in solution while capturing a human polyclonal antibody (e.g., human polyclonal antibody ChromPure Human IgG) of 534 nm or less, and/or a plasmon wavelength of the nanoparticle in solution while capturing NIST monoclonal antibody (reference material RM8671) mAb of 533 nm or less.

### 3. NANOPARTICLES

In a second aspect, the disclosure relates to a nanoparticle comprising on the surface a capture agent and a positively-charged polymer.

In some embodiments, the nanoparticle is a metal nanoparticle, specifically a gold nanoparticle.

In some embodiments, the capture agent and/or the positively-charged polymer are conjugated to the nanoparticle. The amount of positively-charged polymer (by mass of positively-charged polymer and capture agent) can be as described above. In a specific embodiment, the nanoparticle is a stable nanoparticle, i.e. stabilized, for example according to the use of the first aspect. Specifically, the plasmon wavelength of the nanoparticle in solution while capturing a human polyclonal antibody (e.g., human polyclonal antibody ChromPure Human IgG) is 534 nm or less, and/or the plasmon wavelength of the nanoparticle in solution while capturing NIST monoclonal antibody (reference material RM8671) mAb is 533 nm or less.

### 4. SOLUTIONS COMPRISING NANOPARTICLES

In a third aspect, the disclosure relates to a solution comprising a plurality of the nanoparticle of the second aspect. As such, the solution may also be termed a nanoparticle suspension.

In some embodiments, the solution is a buffer, specifically of a low ionic strength. The pH of the solution/buffer may be 3.5-7 (e.g., 4.5-7), specifically 4-6.5 or 4-6. A plurality means at least 2, at least 10, at least 100, or at least 1000.

It is to be understood that wherever this disclosure refers to a use of a nanoparticle of the second aspect, it is intended to also refer to the use of the solution of the third aspect instead.

### 5. KITS COMPRISING NANOPARTICLES

In a fourth aspect, the disclosure relates to a kit comprising a nanoparticle (in particular a plurality thereof) and a positively-charged polymer.

The kit may further comprise a solution like a buffer, in particular a low ionic strength solution/buffer. The pH of the solution/buffer may be 3.5-7 (e.g., 4.5-7), specifically 4-6.5 or 4-6. In further embodiments, the kit may also comprise (i) a capture agent, optionally conjugated to the nanoparticle, and/or (ii) a panel of at least 2, e.g., at least 3, 4, 5 or 6, calibration proteins. The kit may in addition comprise instructions for use providing the plasmon wavelength of the calibration proteins of the panel of calibration proteins. These plasmon wavelengths are known or “historical” plasmon wavelengths of the calibration proteins.

### 6. METHODS FOR DETERMINING SELF-ASSOCIATION OF A TARGET PROTEIN

In a fifth aspect, the disclosure relates to a method for determining the tendency of a target protein to self-associate, comprising the steps of
(i) capturing the target protein in a solution as defined in the third aspect,
(ii) determining the color of the solution, wherein a change in the color of the solution compared to a control solution as defined in the third aspect without a target protein indicates a tendency of the target protein to self-associate.

The control solution may be the solution before step (i) and before the target protein is added, or it may be a separate solution (e.g., undergoing the same method with the exception of step (i)).

The concentration of the target protein may be as described above for target protein analysis. The step of capturing may comprise mixing and/or incubating (e.g., as described below with regard to the sixth aspect) the solution.

The change in color is usually towards a higher absorption wavelength. In some embodiments, determining the color comprises determining the light absorption, wherein a change in the light absorption compared to the control solution indicates a tendency of the target protein to self-associate. In specific embodiments, step (ii) comprises determining the plasmon wavelength, wherein a change in the plasmon wavelength compared to the control solution indicates a tendency of the target protein to self-associate. The determination may comprise measuring the absorbance of light at multiple wavelengths ranging from 450 nm to about 750 nm to determine the plasmon wavelength. The wavelength corresponding to maximal absorbance (plasmon wavelength) shifts to greater values as the separation distance between nanoparticles is reduced. Thus, as the target protein self-associates, the absorbance spectrum of the nanoparticles changes and the plasmon wavelength increases. For example, when the nanoparticle is a gold nanoparticle, a color change towards red, e.g., a red shift in plasmon wavelength indicates a tendency of the target protein to self-associate. Usually, the extent of the change of color, light absorption or plasmon wavelength, respectively, indicates the strength of the tendency of the target protein to self-associate. However, in some embodiments, the change can be used as a binary indication of self-association and no self-association, based on whether the plasmon wavelength changes due to capturing of the target protein.

The solution is usually a solution as defined in the third aspect. The nanoparticle in solution (or the solution) has, in a select embodiment, a plasmon wavelength while capturing a human polyclonal antibody (e.g., human polyclonal antibody Chrom Pure Human IgG) of 534 nm or less, and/or a plasmon wavelength while capturing NIST monoclonal antibody (reference material RM8671) mAb of 533 nm or less. To this end, the method may comprise prior to step (i) a step of selecting a nanoparticle (or solution), wherein the plasmon wavelength of the nanoparticle in solution while capturing a human polyclonal antibody (e.g., human polyclonal antibody ChromPure Human IgG) is 534 nm or less, and/or the plasmon wavelength of the nanoparticle in solution while capturing NIST monoclonal antibody (reference material RM8671) mAb is 534 nm or less, for the capturing in step (i).

In some embodiments, the method comprises a step of calibrating using a panel of at least 2, preferably at least 3, 4, 5 or 6, calibration proteins (e.g., antibodies, specifically monoclonal antibodies) having different tendencies to self-associate. These tendencies are known. The calibrating may comprise the steps of:
(i) measuring the plasmon wavelength of each calibration protein of the panel,
(ii) calculating a historical CS-SINS score for each calibration protein (cP) according to the following formula: CS-SINS Score=(cP—parameter 1)/(parameter 2—parameter 1), wherein parameter 1 is the plasmon wavelength of the calibration protein with the lowest tendency to self-associate of the panel and parameter 2 is the plasmon wavelength of the calibration protein with the highest tendency to self-associate of the panel,
(iii) calculating a new CS-SINS score for each calibration protein according to the following formula: CS-SINS Score=(cP—parameter 1)/(parameter 2—parameter 1), wherein parameter 1 is the plasmon wavelength of the calibration protein with the lowest tendency to self-associate of the panel and parameter 2 is the plasmon wavelength of the calibration protein with the highest tendency to self-associate of the panel, and fitting the parameters to maximize the agreement of the linear fit between the historical and new parameters by minimizing the following term: ((1-slope)²+(intercept)²)),
(iv) determining whether the calibration meets the following criteria a)-c) for a linear fit between the new and the historical data:
   a) slope of linear fit is between 0.9 and 1.1,
   b) intercept of linear fit id between −0.1 and 0.1,
   c) R² of linear fit is above 0.9; and
(v) if all criteria of step (iv) are met, calibrating the CS-SINS score of the target protein using the fitted parameters identified in step (iii). Step (ii) can be carried out before, after or at the same time as step (i). In a specific embodiment, a target protein CS-SINS score of less than or equal to about 0.35 indicates that the target protein has a low tendency to self-associate (i.e. has favorable self-association properties for formulations and compositions comprising high concentrations of the target protein (e.g., greater than or equal to 100 mg/mL target protein)).

### 7. METHODS FOR SCREENING A TARGET PROTEIN

The present disclosure provides methods for screening a target protein in dilute concentrations for self-association properties (e.g., opalescence and viscoelastic properties). Generally, the use of the method of the fifth aspect is provided for screening target proteins for their tendency to self-associate. More specifically, in a sixth aspect, the disclosure relates to methods that may comprise adsorbing a capture agent and a positively-charged polymer onto a nanoparticle (e.g., a metal nanoparticle) to form a capture agent conjugate; incubating the capture antibody conjugate with the target protein in a solution (e.g., buffer) to form a target protein conjugate; measuring the absorbance of light of the target protein conjugate at multiple wavelengths ranging from 450 nm to about 750 nm; and identifying a plasmon wavelength as the wavelength at which there is maximal absorbance by the target protein conjugate.

The positively-charged polymer is co-adsorbed onto a surface (e.g., nanoparticle) with the capture agent

The methods may comprise incubating the capture antibody conjugate with the target protein in a solution (e.g., buffer) to form a target protein conjugate. The incubation may be any length of time necessary to allow binding of the target protein to the capture agent, thus forming the target protein conjugate. For example, the incubation may be at least 30 minutes, at least 1 hour, at least 2 hours, at least 4 hours, at least 5 hours, at least 6 hours, at least 8 hours, at least 10 hours or more.

The buffer used for the incubation can have any desired properties (pH, ionic strength, etc.) but usually mimics the buffer conditions desired for the eventual downstream use of the target protein. In some embodiments, the buffer conditions are those which are physiologically tolerated in a therapeutic. In some embodiments, the pH of the buffer, and by extension the incubation, is between 4.5 and 7. The pH of the buffer may be about 4.5, about 5, about 5.5, about 6, about 6.5 or about 7. In some embodiments, the buffer has low ionic strength.

The methods may comprise measuring the absorbance of light of the target protein conjugate at multiple wavelengths ranging from 450 nm to about 750 nm and identifying a plasmon wavelength as the wavelength at which there is maximal absorbance by the target protein conjugate. The plasmon wavelength (λ_{ρ}), i.e., the wavelength corresponding to maximal absorbance shifts to greater values as the separation distance between nanoparticles is reduced. Thus, as the target protein engages in self-association, the absorbance spectrum of the nanoparticles changes and that is reflected in the identification of the plasmon wavelength. In some embodiments, this change can be used as a binary indication of self-association and no self-association, based on whether the plasmon wavelength changes upon absorbance measurements of the target protein conjugates relative to the capture agent conjugates.

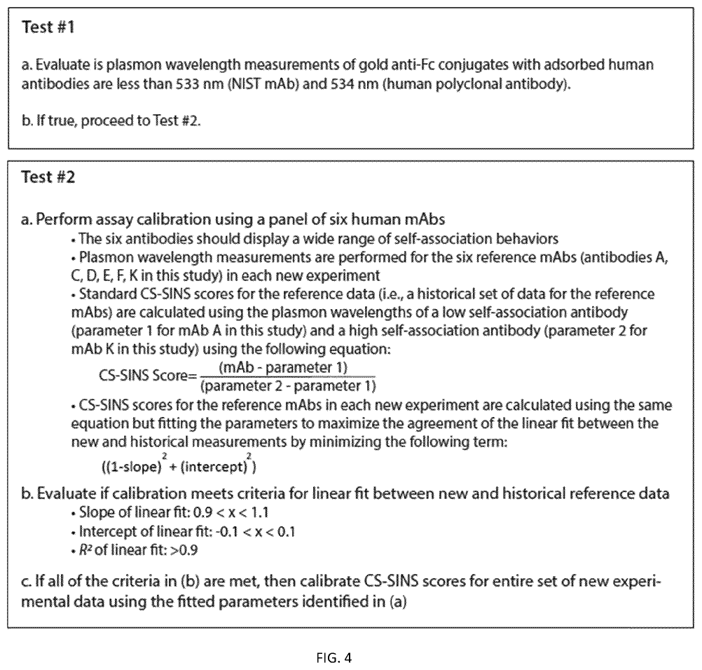

The sensitivity of the plasmon wavelength to changes in the interparticle distances between antibody-gold conjugates can be used as a measure of the extent or degree of self-association of the target protein. In some embodiments, the methods further comprise calculating charge-stabilized self-interaction nanoparticle spectroscopy (CS-SINS) score. The CS-SINS score is a ratio of the plasmon wavelength of the target protein conjugate with a subtraction of a plasmon wavelength from a low self-association control protein conjugate to a difference of plasmon wavelength from a high self-association control protein conjugate and the low self-association control protein conjugate, as described in FIG. 4 (Test #2) and shown in Equation (1):

$$CS - SINS Score = \frac{( mAB - parameter 1 )}{( parameter 2 - parameter 1 )} \qquad ( 1 )$$

wherein parameter 1 is the plasmon wavelength of a low self-association antibody and parameter 2 is the plasmon wavelength of a high self-association antibody.

In some embodiments, a CS-SINS score less than or equal to about 0.35 indicates that the target protein has favorable self-association properties for formulations and compositions comprising high concentrations of the target protein (e.g., greater than or equal to 100 mg/mL target protein).

In some embodiments, the method further comprises a calibration method comprising: capturing a plurality of calibration proteins with the capture agent conjugate to form a plurality of calibration protein conjugates; measuring the absorbance of light of each of plurality of calibration protein conjugates at multiple wavelengths ranging from 450 nm to about 750 nm; and identifying the plasmon wavelength.

The calibration method may further comprise: calculating a CS-SINS score for each of plurality of calibration protein conjugates; and comparing the CS-SINS scores for each of plurality of calibration protein conjugates with previous calibration protein conjugate CS-SINS scores for linear fit. For example, CS-SINS for each of the plurality of the calibration protein conjugates are fit to maximize the agreement of the linear fit between the new data and previous CS-SINS scores by minimizing the following term: ((1−slope)²+(intercept)²). The calibration meets the criteria for a linear fit between the new and previous data and, thus, proper calibration if:
the slope of the linear fit=0.9<x<1.1;
the intercept of linear fit=−0.1<x<0.1; and
R² of linear fit=>0.9.

The plurality of calibration proteins comprises proteins with a range of self-association properties (e.g., opalescence and viscoelastic properties). The calibration proteins may be the same type of protein as the target protein. For example, if the target protein is antibody, the calibration proteins are selected from different antibodies with varying self-association properties. In some embodiments, each of the plurality calibration proteins are each individually selected from the group consisting of a monoclonal antibody, a polyclonal antibody, and fragments or derivatives thereof.

In some embodiments, the calibration proteins include the low self-association control protein and the high self-association control protein, which the plasmon wavelength of conjugates thereof are used to calculate a CS-SINS, as described elsewhere herein.

In some embodiments, the plurality of calibration proteins comprises NIST Reference Antibody RM 8671 and human polyclonal antibody ChromPure Human IgG, whole molecule. In some embodiments, the calibration identifies the plasmon wavelength of the NIST Reference Antibody RM 8671 and the human polyclonal antibody ChromPure Human IgG, whole molecule. A proper calibration would comprise a plasmon wavelength of the NIST Reference Antibody RM 8671of less than 533 nm and a plasmon wavelength of the human polyclonal antibody ChromPure Human IgG, whole molecule of less than 534 nm.

### 8. SYSTEMS OR KITS FOR SCREENING A TARGET PROTEIN

In a seventh aspect, the disclosure relates to systems or kits (e.g., reagents, computer software, instruments, etc.) for screening a target protein in dilute concentrations for favorable self-association properties (e.g., opalescence and viscoelastic properties). In some embodiments, the systems comprise one or more or each of a capture agent, a positively-charged polymer, a surface (e.g., metal nanoparticle), a solution (e.g., buffer), a target protein, at least one calibration protein, and a spectrophotometer. The descriptions provided above for the capture agent, positively-charged polymer, surface (e.g., metal nanoparticle), solution (e.g., buffer), and target protein provided elsewhere herein are also applicable to the disclosed systems. The spectrophotometer can include any variety of instruments that measure light absorption and/or transmission at a variety of light wavelengths. Individual member components of the systems or kits may be physically packaged together or separately.

The systems can also comprise instructions for using the components of the systems. The instructions are relevant materials or methodologies pertaining to the systems. The materials may include any combination of the following: background information, list of components and their availability information (purchase information, etc.), brief or detailed protocols for using the systems, trouble-shooting, references, technical support, and any other related documents. Instructions can be supplied with the systems or as a separate member component, either as a paper form or an electronic form which may be supplied on computer readable memory device or downloaded from an internet website, or as recorded presentation.

It is understood that the disclosed systems and kits can be employed in connection with the disclosed methods.

### 9. EXAMPLES

### Materials and Methods

Immunogold conjugate preparation for AC-SINS in PBS Goat-antihuman Fcγ-specific antibody (Jackson ImmunoResearch Laboratories, 109-005-008) was buffer exchanged twice using Zeba desalting columns (Thermo Fisher Scientific, PI-89882) into 20 mM acetate (pH 4.3). The concentration was determined using UV absorbance at 280 nm and a mass extinction coefficient of 1.26 mL/mg*cm. The antibody was diluted to 0.4 mg/mL in 20 mM acetate (pH 4.3). One milliliter of 20 nm gold nanoparticles (7.0×10¹¹ particles/mL; Ted Pella Inc., 15705) was sedimented in 1.5 mL microcentrifuge tubes (1615-5500, USA Scientific) at 21130 rcf for 6 min. Next, 950 μL of supernatant were removed and replaced with 950 μL milliQ water. The resuspended particles were further diluted by adding 500 μL of milliQ water (final concentration of 4.67×1011 particles/mL). To 100 μL of prepared capture antibody, 900 μL of gold nanoparticles were added. The mixtures were incubated at room temperature overnight. Prior to use, the immunogold conjugates were sedimented at 21130 rcf for 6 min. Nine hundred fifty microliters of supernatant were removed and reserved, and the particles were resuspended in the remaining supernatant. The volume was carefully readjusted to 50 μL.

Preparation of polylysine-stabilized immunogold conjugates for CS-SINS Goat-antihuman Fcγ-specific antibody (Jackson ImmunoResearch Laboratories, 109-005-008) was buffer exchanged as described above and diluted to a final concentration of 0.8 mg/mL in 20 mM acetate (pH 4.3). Polylysine (Fisher Scientific, ICN19454405), initially dissolved in milliQ water at 5 mg/mL was diluted to either 0.8 mg/mL (0.03 polylysine/IgG fraction), 2.67 mg/mL (0.10 polylysine/IgG fraction), or 4.0 mg/mL (0.15 polylysine/IgG fraction) in 20 mM acetate (pH 4.3). For a 100 μL conjugate preparation, 48.5 μL of capture IgG was mixed with 1.5 μL of polylysine and reserved. Twelve hundred microliters of 20 nm gold nanoparticles (Ted Pella, 15705) were sedimented at 21130 rcf for 6 min, after which 1150 μL of supernatant was removed. The nanoparticles were resuspended in the remaining supernatant and the volume was carefully adjusted to 50 μL. The nanoparticles were then added to the IgG/polylysine mixture and rapidly mixed by pipetting up and down 20 times. The conjugates were incubated at room temperature overnight. The described preparation is for 100 μL of conjugates but can be scaled to prepare larger volumes if desired. In experiments where non-capture antibody was used, goat polyclonal IgG (Jackson ImmunoResearch Laboratories, 005-000-003) was substituted for goat-antihuman IgG.

Dynamic light scattering and zeta potential measurements of immunogold conjugates Dynamic light scattering (DLS) experiments were performed using the Zetasizer Nano ZSP (Malvern Panalytical, Worcestershire, UK). To assess the size of immunogold conjugates in a given buffer, 950 μL of buffer were added to 50 μL of the conjugates and immediately transferred to folded-capillary zeta cells (DTS1070, Malvern Panalytical). The cuvettes were transferred to the Zetasizer instrument and the size was measured at 25° C. using DLS with 173° backscatter measurements. The size measurements were an average of at least thirty 10 s measurements. In cases where the samples were monodisperse (immunogold conjugates prepared for standard AC-SINS implementations), z-the average diameter calculated using cumulants analysis was reported. In cases where the samples were polydisperse (polylysine stabilized immunogold conjugates), the major peak of the size distribution as determined by non-negative least squares analysis was reported. Immediately following particle sizing, the zeta potential of the particles was measured using laser doppler velocimetry on the same instrumentation. Data was collected and analyzed using Zetasizer 7.11 Software (Malvern Panalytical).

CS-SINS assay in histidine Antibodies of interest were buffer exchanged twice using Zeba desalting columns (Thermo Fisher Scientific, P189882) into 10 mM histidine (pH 6.0). The concentration was determined using UV absorbance at 280 nm and a mass extinction coefficient uniquely calculated for each monoclonal antibody [1.40 m L/mg*cm was used for human polyclonal antibody (Jackson ImmunoResearch Laboratories, 009-000-003)]. The antibodies were diluted to 11.1 μg/mL in histidine (pH 6.0) prior to use in CS-SINS. To a transparent and flat-bottom 384-well polystyrene plate (Thermo Fisher Scientific, 12565506), 5 μL of immunogold conjugate were added in triplicate for each mAb assayed and for human polyclonal antibody. Next, 45 μL of each antibody was added to the immunogold conjugates and mixed by pipetting up and down 10 times (final antibody concentration of 10 μg/mL). The mixtures were covered and incubated at room temperature for 4 h, after which the absorbance was measured from 450-650 nm in 1 nm increments (normal scanning speed, 8 reads per well) using a BioTek Synergy Neo plate reader (BioTek, Winooski, Vt.). The plasmon wavelength was determined by fitting a second order polynomial to 40 data points around the observed maximum absorbance and setting the first derivative to 0. The CS-SINS score was calculated as described below.

Calibration of CS-SINS assay The CS-SINS measurements were calibrated and evaluated in the following manner. First, each new batch of gold-anti Fc conjugates was evaluated using two control antibodies, namely human polyclonal antibody (ChromPure Human IgG, whole molecule, Jackson ImmunoReasearch Laboratories 009-000-003) and the NIST mAb RM 8671 (NIST mAb) (See, Karageorgos, I., et al., Biologicals 2017, 50, 27-34, incorporated herein by reference in its entirety). If the plasmon wavelengths for these conjugates with adsorbed human antibody were greater than 534 nm (human polyclonal antibody) or 533 nm (NIST mAb), the experiments were terminated and the conjugates were prepared again until they passed this test (Test #1). Second, the gold-anti-Fc conjugates that pass Test #1 were then evaluated using a panel of six control mAbs that spanned both low and high self-association behaviors (mAbs A, C, D, E, F and K in this study), and their plasmon wavelengths (in the form of CS-SINS scores) were compared to reference data for the same antibodies. For the reference data, the CS-SINS scores were calculated as: (plasmon wavelength for a given mAb—plasmon wavelength for the lowest self-association mAb such as mAb A in this study)/(plasmon wavelength for the highest self-association mAb such as mAb K in this study—plasmon wavelength for the lowest self-association mAb such as mAb A in this study). For the measurements of the panel of mAbs in each new experiment (i.e., the calibration measurements), the CS-SINS scores were calculated as: (plasmon wavelength for a given mAb—parameter 1)/(parameter 2—parameter 1). The two parameters were fit to minimize the sum of ((1-slope)²+(intercept)²), where the slope and intercept terms are obtained from the linear regression between the calibration and reference CS-SINS Scores. If any of the three values of slope, intercept or R² values for this linear fit were not within 10% of the ideal values (1 for slope, 0 for intercept and 1 for R²), the experiment was terminated and conjugates were prepared again until they passed this test (Test #2) in addition to Test #1. If the measurements pass both tests, then the CS-SINS measurements were performed on the full panel of antibodies and the CS-SINS scores were calculated using the parameters fit in Test #2 (i.e., parameters 1 and 2). The calibration antibodies include: A, tocilizumab; C, cetuximab; D, evolocumab; E, denosumab; F, pembrolizumab; and K, omalizumab.

Viscosity and opalescence measurements The viscosity and opalescence values for the experimental mAb dataset were obtained from Kingsbury et al. (Sci Adv 6, eabb0372, doi:10.1126/sciadv.abb0372 (2020), incorporated herein by reference in its entirety).

### Example 1

### Charge Stabilization Prevents Aggregation of Antibody-Gold Conjugates

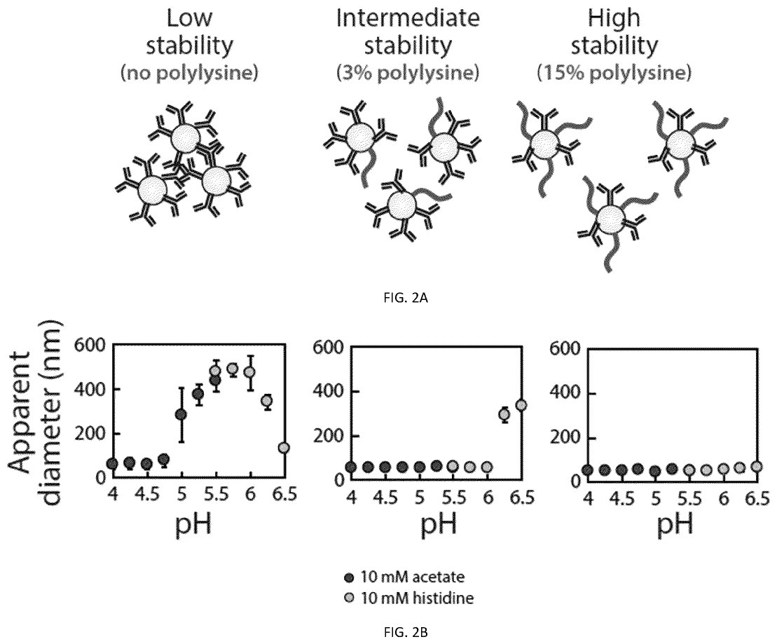

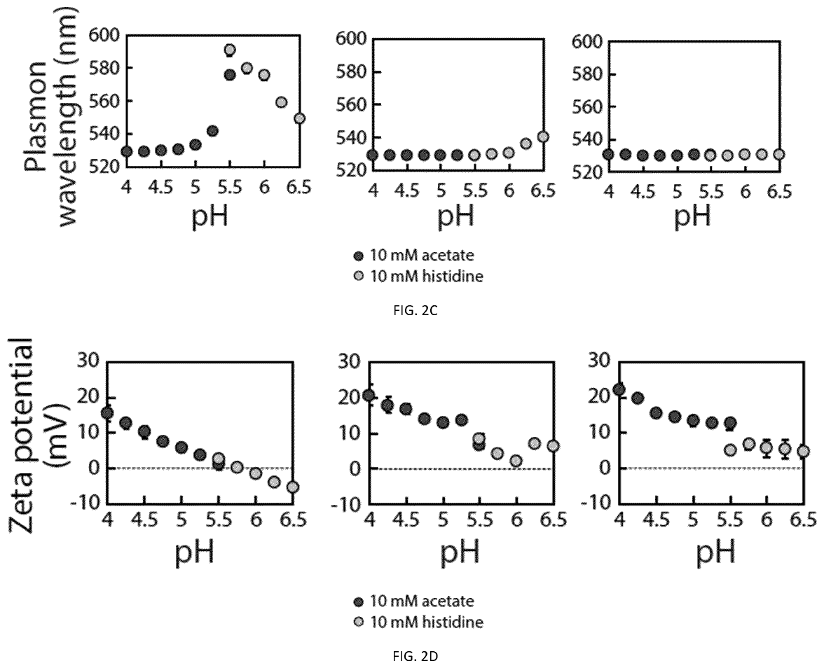

To understand the origins of the problems experienced in previous attempts to use AC-SINS (FIG. 2A, left), the aggregation of gold-antibody (anti-human IgG) conjugates was measured in dilute buffers (10 mM acetate and/or histidine) over a range of pH values (pH 4.0-6.5; FIG. 2B, left). For pH values in the range of 5.0-6.5, plasmon wavelengths as high as ˜590 nm were observed, a significant departure from typical values for stable conjugates (530-535 nm) in PBS (FIG. 2E). Substantial particle aggregation was observed in the range of pH 5.0-6.5 as evidenced by the large apparent diameter of the conjugates detected by dynamic light scattering. Consistent with this result, plasmon wavelengths as high as ˜590 nm were observed, a significant departure from typical values for stable conjugates (530-535 nm) in PBS (FIG. 2C, left). The mechanism of particle aggregation was investigated by measuring the zeta potential of the antibody-gold conjugates (FIG. 2D, left) and a steady decrease was observed from a maximum value +16 mV at pH 4.0, crossing zero near pH 5.7 with a minimum of −5 mV at pH 6.5. The trend in zeta potential with increasing pH was also consistent with the titration of charged amino acids intrinsic to the immobilized capture antibody. These findings suggested that low net charge of the conjugates near pH 5.5-6 was linked to their aggregation in the pH 5-7 range.

A large, positively-charged polymer (polylysine, ≤70 kDa) was co-adsorbed with the capture antibody during conjugate preparation. Addition of even small amounts of polylysine (e.g., 3%, FIG. 2A, middle) was sufficient to prevent conjugate aggregation over a broad pH range of 4 to 6 (FIG. 2B, middle). Increasing the polylysine to 15% (FIG. 2A, right) led to further stabilization to pH 6.5 (FIG. 2B, right). The plasmon wavelengths of the conjugates stabilized with polylysine displayed similar pH-dependent trends as observed for the apparent sizes of the conjugates (FIGS. 2C, middle and 2C, right). A concomitant increase in zeta potential of the conjugates to positive values (+2 to +6 mV) was also observed over this same pH range (FIGS. 2D, middle and 2D, right). The fact that low amounts of polylysine (3%) yielded a zeta potential at pH 6 (+2.3mB) that would be associated with aggregation of conjugates in the absence of polylysine suggested an additional, steric component to the observed stabilization.

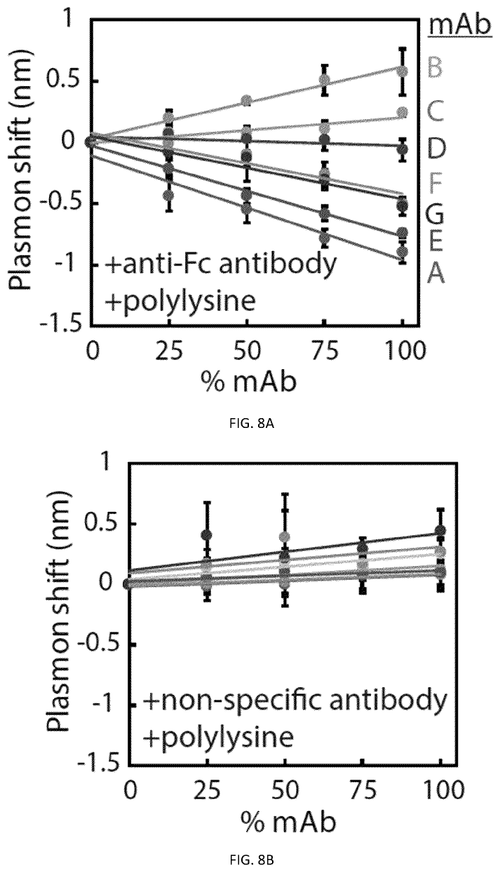

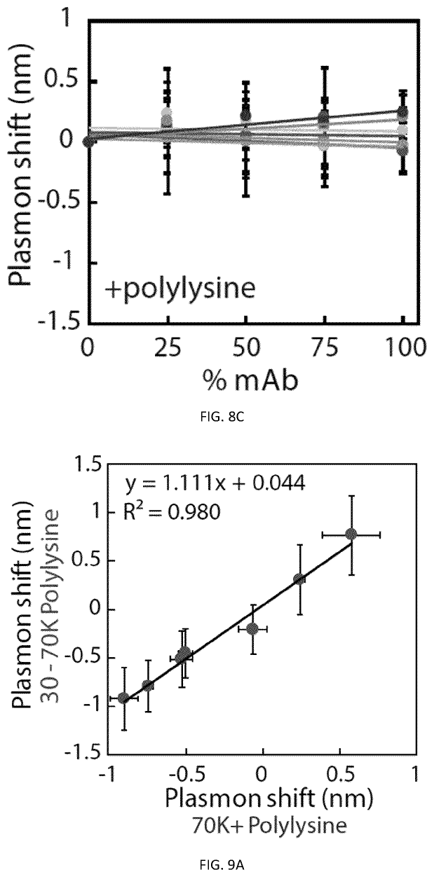

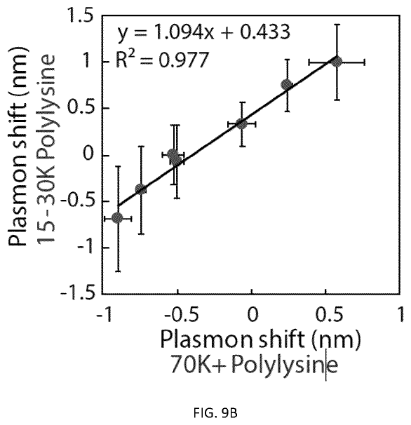

Stabilized conjugates were used to measure mAb self-interactions via CS-SINS in histidine formulations (FIG. 8). Seven mAbs that display a wide range of self-association behaviors based on light scattering analysis of mAb colloidal interactions in histidine formulations were selected. CS-SINS analysis revealed small but detectable, mAb-dependent plasmon shifts—plasmon shifts of less than <1 nm compared to greater than 30 nm observed for the same antibodies using conventional AC-SINS in PBS. The capture antibody was substituted with a non-specific antibody and even smaller plasmon shifts were observed for the seven mAbs. Conjugates prepared with only polylysine prior to addition of mAbs also resulted in extremely small plasmon shifts that were mAb independent. The impact of polylysine size (nominal sizes of 30-70 kDa and 15-30 kDa) on the plasmon shifts was tested and similar trends were observed for the conjugates prepared with different sizes of polylysine (FIG. 9). These results indicated that the CS-SINS assay detects mAb-specific colloidal interactions that require antibody-mediated immobilization and depend weakly on polylysine size.

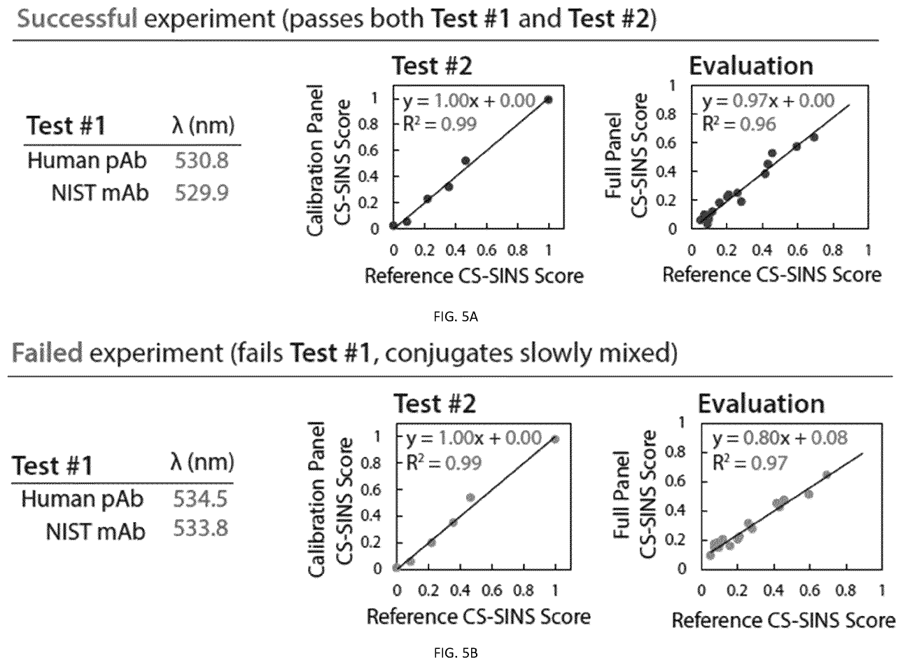

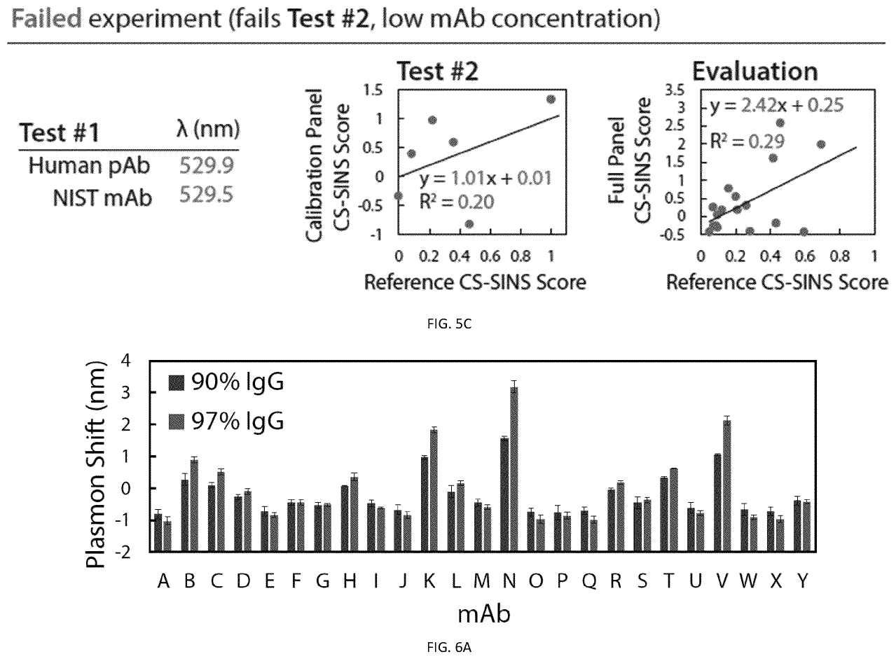

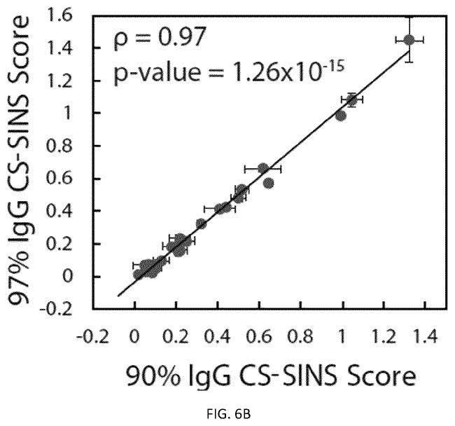

Robust calibration methods control for the variability of results that are possible if precise experimental protocols are not followed. Two tests for calibrating and evaluating CS-SINS measurements were developed (FIGS. 4 and 5). 1) Anti-Fc gold conjugates for two calibration antibodies human polyclonal antibody and NIST mAb should result in plasmon wavelengths of <534 nm for human polyclonal antibody and <533 nm for the NIST mAb, as shown in FIG. 5A for a successful experiment. 2) A panel of six calibration mAbs were used to calibrate the assay and these measurements were compared to reference (historical) measurements obtained in multiple independent assays. Two parameters were fit to maximize the linear fit between the calibration and reference data, as explained in the Methods section and FIG. 4, to transform the plasmon wavelengths into calibrated CS-SINS scores. If the slope, intercept and R2 values were within 10% of their ideal values, then the assay passed Test #2 and the full panel of antibodies was evaluated. For the successful experiment in FIG. 5, the assay passed Test #2 and the evaluation data for the larger panel of antibodies (without the calibration antibodies) showed good performance relative to reference data. The calibration protocol was applied to CS-SINS measurements made with either 3% or 10% polylysine for a panel of 25 commercial mAbs (FIG. 6A). When analyzed as plasmon wavelength shifts, there were obvious differences in measurements between the two polylysine conditions. However, when the calibration protocol was applied, there was excellent agreement between the two datasets (FIG. 6B). To illustrate the value of this calibration process, additional experiments were performed to illustrate how experiments with flawed protocols can be readily identified (FIGS. 5B-5C).

### Example 2

### mAb Self-Interaction as Prediction of Solution Behavior

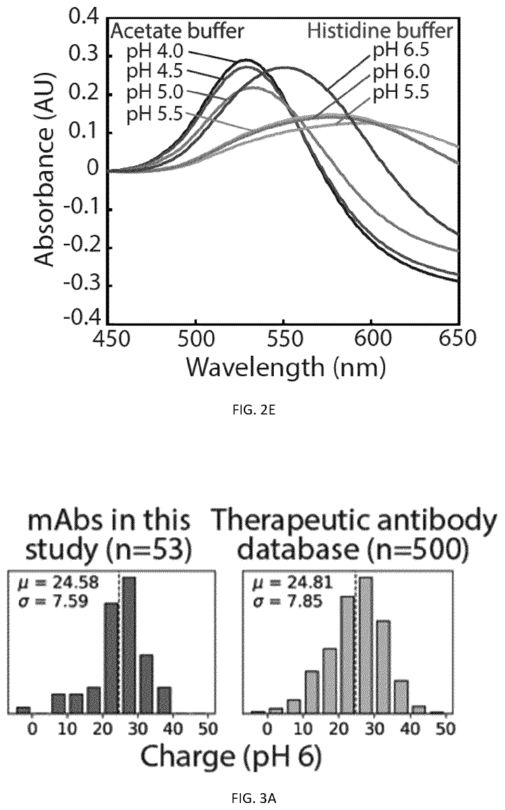

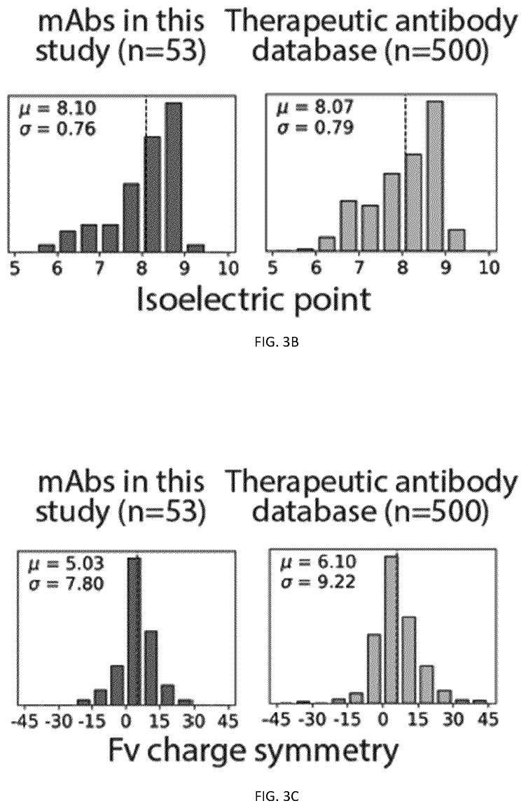

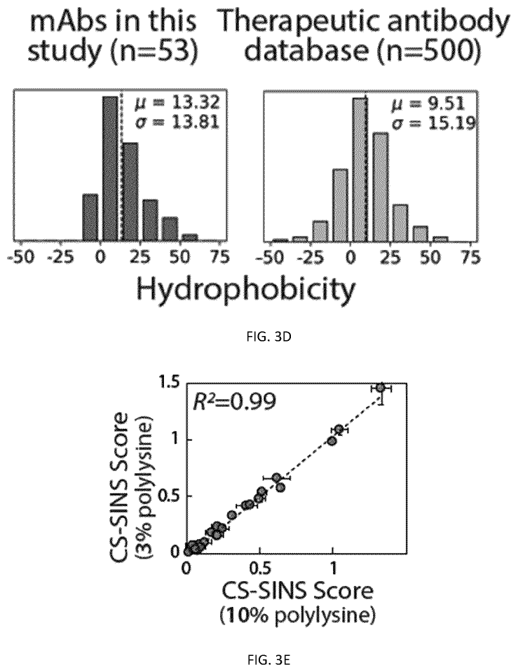

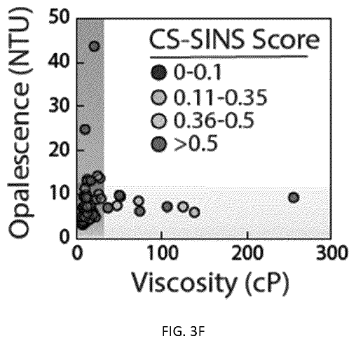

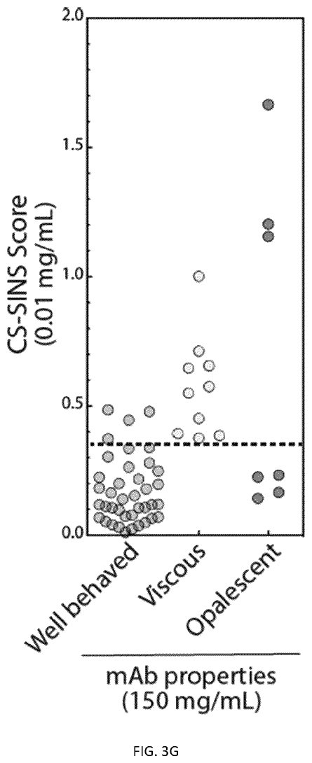

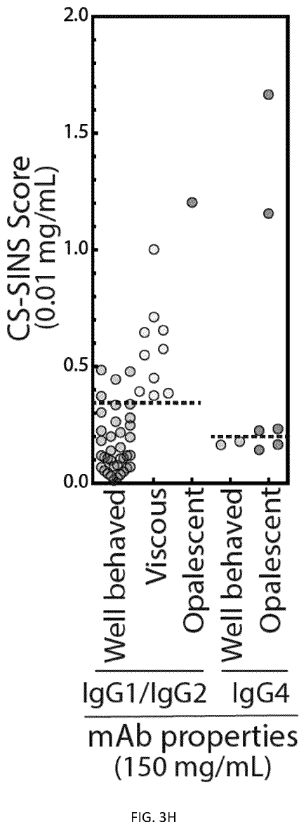

To assess the utility of charge-stabilized immunogold conjugates in measuring mAb self-interaction and consequently predicting antibody solution behavior, a diverse set of 56 mAbs, including 43 commercial products, was employed. A range of amino acid sequence-related properties (pl, charge, charge asymmetry, and hydrophobicity) were compared for mAbs within the dataset to 500 unique antibody sequences, drawn from the broader clinical landscape using the Therapeutic Antibody Database (TABS) (FIGS. 3A-D). Emphasis was placed on evaluating sequence-derived metrics of electrostatic and hydrophobic properties, both of which have been shown previously to be drivers of mAb self-interaction. Based on this analysis, the mAb set was demonstrated to be a faithful representation of key properties of mAbs from the broader clinical antibody landscape, including their isoelectric points, net charges (pH 6) and Fv charge asymmetry parameters (Fv-CSP, pH 6). Moreover, the panel of antibodies also displayed similar hydrophobicity properties as evaluated in terms of the Eisenberg Hydrophobicity Index (EHI), relative to the large pane of clinical-stage antibodies. The findings indicated that the mAb dataset was well suited for evaluating the general applicability of the CS-SINS assay for evaluating antibody solution behavior.

Greater than 30% of clinical-stage mAbs, and likely a higher percentage in earlier preclinical stages, can be expected to exhibit poor solution behavior such as high solution viscosity and opalescence, which manifest only at higher concentrations (>100 mg/mL). High mAb solution viscosity is particularly problematic for subcutaneous delivery via auto-injectors, and during ultra/diafiltration purification unit operations. High opalescence can indicate predisposition for phase separation and aggregation. It was recently shown that measurement of weak, colloidal self-interactions via the diffusion interaction parameter (k_{D}) was most effective in predicting mAbs prone to high viscosity or opalescence relative to a large set of molecular descriptors. However, the material requirements for such measurements render them to be implementable only at the later, candidate selection stages of the discovery process (FIG. 1) with only a few (˜10) candidates. The CS-SINS assay, on the other hand, which also measures weak colloidal interactions, is amenable to implementation during candidate optimization for screening 100s to 1000s of variants (FIG. 1).

CS-SINS was conducted in ultra-dilute solutions (10 μg/ml), to determine if it could detect problematic behaviors that were manifested at four orders of magnitude higher antibody concentrations (>100 mg/ml). Viscous (>30 cP) antibodies consistently had higher CS-SINS scores than well-behaved mAbs with low viscosity and that mAbs with extreme opalescence profiles (>20 NTU) were easily identified using CS-SINS (FIG. 3F). With a threshold value of 0.35, CS-SINS identified well-behaved mAbs (<30 cP, <12 NTU) with >85% accuracy (FIG. 3G-3H). Setting this CS-SINS score threshold facilitated the identification of all 10 (100%) viscous antibodies and 3 of 7 (43%) opalescent antibodies. Additionally, this threshold only les to the mischaracterization of 4 of the remaining 39 (10%) well-behaved antibodies. Of the 56 mAbs, 35 of 39 were correctly identified as well-behaved and 13 of 17 were identified as having poor solution properties, comparable to classification by kip. The CS-SINS assay was also robust with respect to reproducibility, supported by detailed calibration and system suitability criteria (FIGS. 3E, 4, and 5).

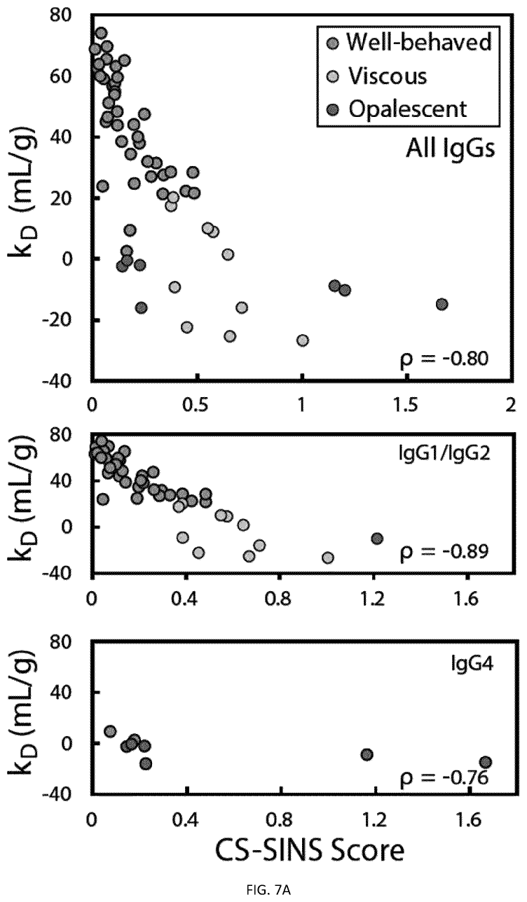

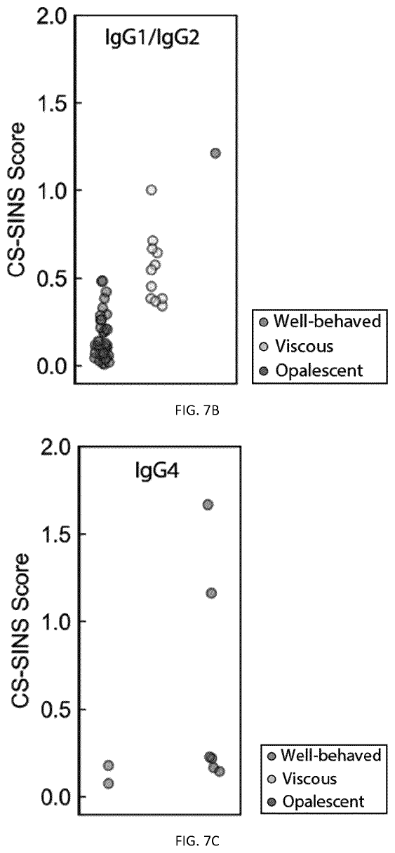

Previous work using a similar panel of antibodies revealed a strong relationship between the diffusion interaction parameter (kip) and poor solution properties. Further, as identified herein IgG subclass-specific behavior with respect to the utility of kip in predicting poor solution properties, particularly with respect to opalescence. The relationship between kip and the CS-SINS scores measured herein was explored (FIG. 7). Without segregating mAbs by subclass, a moderately strong rank correlation was observed between kip and CS-SINS score (Spearman's ρ of −0.80; FIG. 7A, top). However, when the mAbs were divided into IgG1/IgG2 versus IgG4 subclasses, subclass-specific behavior emerged. There was a marked increase in the rank correlation for the IgG1/IgG2 grouping (Spearman's ρ of −0.89; FIG. 7A, middle) and a decrease for the IgG4 subclass (Spearman's ρ of −0.76; FIG. 7A, bottom). Extending this analysis to the prediction of poor solution behaviors, it was observed that using a CS-SINS threshold of 0.35 facilitated the detection of all poorly behaved molecules in the IgG1/IgG2 subclass grouping (FIG. 7B). Applying the same standard to the IgG4 grouping revealed that all of the opalescent molecules with low CS-SINS scores belonged to this subclass (FIG. 7C), suggesting that the mode of IgG4 self-association leading to its opalescent behavior may be unique relative to IgG1/2 antibodies. More generally, these results collectively demonstrated the ability of CS-SINS to identify antibodies with high levels of viscosity and opalescence, especially for IgG1 and IgG2 mAbs, using ultra-dilute (10 μg/mL) solution measurements of self-association. The assay is robust and readily implementable for simultaneous, high-throughput screening of large numbers (˜100-1000) of mAb candidates. The CS-SINS assay represents a major advance towards identification of developable antibodies with drug-like properties in early discovery.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A method of stabilizing a nanoparticle comprising a capture agent on the surface, the method comprising conjugating a positively-charged polymer to the nanoparticle, wherein the positively-charged polymer is a homopolymer of a positively charged amino acid or a heteropoiymer of two or more positively charged amino acids.

2. The method of claim 1, wherein the nanoparticle is stabilized in a solution.

3. A nanoparticle comprising on the surface a capture agent and a positively-charged polymer, wherein the positively-charged polymer is a homopolymer of a positively charged amino acid or a heteropolymer of two or more positively charged amino acids.

4. The nanoparticle of claim 3, wherein the nanoparticle is comprised in a solution.

5. A solution comprising a plurality of the nanoparticle of claim 3.

6. A kit comprising a nanoparticle and a positively-charged polymer, wherein the positively-charged polymer is a homopolymer of a positively charged amino acid or a heteropolyrner of two or more positively charged amino acids.

7. The kit of claim 6, further comprising a solution and/or a capture agent.

8. A method for determining the tendency of a target protein to self-associate, comprising:
(i) capturing the target protein in the solution of claim 5; and
(ii) determining the color of the solution,
wherein a change in the color of the solution compared to a control solution without the target protein indicates a tendency of the target protein to self-associate.

9. A method for screening a target protein in dilute concentrations for self-association properties, comprising:
adsorbing the capture agent and the positively-charged polymer onto the nanoparticle in the kit of claim 7 to form a capture agent conjugate;
incubating the capture agent conjugate with the target protein in a solution to form a target protein conjugate;
measuring the absorbance of light of the target protein conjugate at multiple wavelengths ranging from 450 nm to about 750 nm; and
identifying a plasmon wavelength as the wavelength at which there is maximal absorbance by the target protein conjugate,
wherein the positively-charged polymer is a homopolymer of a positively charged amino acid or a heteropolyiner of two or more positively charged amino acids.

10. A system for screening a target protein in dilute concentrations for self-association properties according to the method of claim 9, said system comprising one or more or each of:
a capture agent;
a positively-charged polymer, wherein the positively-charged polymer is a homopolymer of a positively charged amino acid or a heteropolymer of two or more positively charged amino acids;
a nanoparticle;
a solution;
a target protein;
at least one calibration protein; and
a spectrophotometer.

11. The method of claim 1, wherein the nanoparticle is a metal nanoparticle.

12. The method of claim 1, wherein the positively-charged polymer is polylysine.

13. The method of claim 1, wherein the capture agent is an antibody.

14. The method of claim 8, wherein the target protein is an antibody.

15. The method of claim 2, wherein the solution has a low ionic strength and/or a pH of 3.5-7.

16. The method of claim 11, wherein the metal nanoparticle is a gold particle.
